(19) United States
(12) Patent Application Publication
(10) Pub. No.: US 2017/0216320 A1
(43) Pub. Date: Aug. 3, 2017

(54) ASPIRIN FORMULATION FOR INCREASED EFFICACY

(71) Applicant: Vitalis LLC, Wilmington, DE (US)

(72) Inventor: Joseph Habboushe, New York, NY (US)

(21) Appl. No.: 15/401,912

(22) Filed: Jan. 9, 2017

Related U.S. Application Data
(63) Continuation of application No. 14/739,957, filed on Jun. 15, 2015, now abandoned.
Application No. 14/739,957 is a continuation of application No. 14/023,188, filed on Sep. 10, 2013, now abandoned.

(51) Int. Cl. A61K 31/616; A61K 31/522; A61K 31/167; A61K 9/00; A61K 31/455; A61K 45/06; A61K 9/24

(52) U.S. Cl. CPC A61K 31/616; A61K 45/06; A61K 31/522; A61K 9/209; A61K 9/0056; A61K 31/455; A61K 31/167

(57) ABSTRACT

Provided are methods for enhancing the efficacy of aspirin. Also provided are methods for reducing pain or preventing or treating heart attack, stroke or blood clot in a subject in need thereof. The methods entail orally administering to the subject a first composition comprising a first amount of aspirin, and a second composition comprising a second amount of aspirin, wherein the first composition is formulated so as to, upon administration, disintegrate or dissolve intraorally providing rapid release of the aspirin of the first composition in the subject, and wherein the second composition is formulated to be substantially more difficult than the first composition to disintegrate or dissolve intraorally but is ingestible and releasable in the gastrointestinal track of the subject. The method can further include administering to the subject a painkiller or an agent suitable for treating a cardiovascular disease or condition.

### CROSS-REFERENCE TO RELATED APPLICATIONS

This application is a continuation of U.S. application Ser. No. 14/739,957, filed Jun. 15, 2015, which is a continuation of U.S. application Ser. No. 14/023,188, filed Sep. 10, 2013, which is hereby incorporated by reference in its entirety.

### FIELD OF DISCLOSURE

The present disclosure relates generally to the field of pharmaceutical compositions and therapeutic methods. The compositions include a portion of aspirin for intraoral release such that this portion of aspirin dissolves or disintegrates intraorally, and another portion for gastrointestinal release. The compositions can further include a painkiller or an agent suitable for treating a cardiovascular disease or condition, or any condition that aspirin is suitably used for treating. The compositions and methods disclosed herein are useful for reducing pain, reducing aspirin-related side effects, and/or preventing or treating heart attack, stroke or blood clot.

### BACKGROUND

Aspirin, also known as acetylsalicylic acid, is a salicylate drug, often used as an analgesic to relieve minor aches and pains, as an antipyretic to reduce fever, as an anti-inflammatory medication, as a blood thinner for prevention of cardiovascular disease, to reduce the flush side effect of niacin, and more. Salicylic acid, the main metabolite of aspirin, is an integral part of human and animal metabolism.

Aspirin is part of a group of medications called nonsteroidal anti-inflammatory drugs (NSAIDs), but differs from most other NSAIDs in the mechanism of action. Though it, and others in its group called the salicylates, have similar effects (antipyretic, anti-inflammatory, analgesic) to the other NSAIDs and inhibit the same category of cyclooxygenase enzymes, aspirin (but not the other salicylates) does so in an irreversible manner and, unlike others, affects more the COX-1 variant than the COX-2 variant of the enzyme.

Aspirin also has an antiplatelet effect by inhibiting the production of thromboxane, which under normal circumstances binds platelet molecules together to create a patch over damaged walls of blood vessels. Because the platelet patch can become too large and also block blood flow, locally and downstream, aspirin is also used long-term to help prevent heart attacks, strokes, and blood clots in people at high risk of developing blood clots. It has also been established that aspirin may be given immediately during or after a heart attack or stroke to reduce the mortality and morbidity of that heart attack or stroke, and low dose or regular dose aspirin given on a regular basis to prevent primary and secondary cardiovascular events. Aspirin may be effective at preventing certain types of cancer, particularly colorectal cancer.

The main undesirable side effects of aspirin taken by mouth are gastrointestinal ulcers, stomach bleeding, and tinnitus, especially in higher doses. In children and adolescents, aspirin is no longer indicated to control flu-like symptoms or the symptoms of chickenpox or other viral illnesses, because of the risk of Reye's syndrome.

There is a need, therefore, to provide aspirin formulations that retain its desired therapeutic effect but with reduced side effects.

### SUMMARY

It has been discovered that oral administration of aspirin achieved a remarkably higher therapeutic effect when the aspirin is partially released intraorally and dissolve or disintegrate intraorally and partially released through the gastrointestinal (GI) track, as compared to intraoral or GI release alone.

In accordance with one embodiment of the present disclosure, therefore, provided is a method of increasing aspirin efficacy or reducing aspirin side effects in a subject in need thereof, comprising orally administering to the subject a first composition comprising a first amount of aspirin, and a second comprising a second composition comprising a second amount of aspirin,
wherein the first composition is formulated so as to, upon administration, disintegrate or dissolve intraorally providing rapid release of the aspirin of the first composition in the subject, and
wherein the second composition is formulated to be substantially more difficult than the first composition to disintegrate or dissolve intraorally but is ingestible and releasable in the gastrointestinal track of the subject.

In accordance with another embodiment provided is a method for reducing pain in a subject in need thereof, comprising orally administering to the subject a first composition comprising a first amount of aspirin, and a second composition comprising a second amount of aspirin,
wherein the first composition is formulated so as to, upon administration, disintegrate or dissolve intraorally providing rapid release of the aspirin of the first composition in the subject, and
wherein the second composition is formulated to be substantially more difficult than the first composition to disintegrate or dissolve intraorally but is ingestible and releasable in the gastrointestinal track of the subject.

In accordance with another embodiment provided is a method for preventing or treating preventing or treating heart attack, stroke or blood clot in a subject in need thereof, comprising orally administering to the subject a first composition comprising a first amount of aspirin, and a second composition comprising a second amount of aspirin,
wherein the first composition is formulated so as to, upon administration, disintegrate or dissolve intraorally providing rapid release of the aspirin of the first composition in the subject, and
wherein the second composition is formulated to be substantially more difficult than the first composition to disintegrate or dissolve intraorally but is ingestible and releasable in the gastrointestinal track of the subject.

In accordance with another embodiment provided is a tablet comprising:

a first portion comprising a first amount of aspirin formulated so as to, upon administration to a subject, disintegrate or dissolve intraorally; and
a second portion comprising a second amount of aspirin and a painkiller,
wherein the second portion is formulated to be substantially more difficult than the first portion to disintegrate or dissolve intraorally but is ingestible and releasable in the gastrointestinal track of the subject.

In accordance with another embodiment provided is a tablet comprising:
a first portion comprising a first amount of aspirin formulated so as to, upon administration to a subject, disintegrate or dissolve intraorally; and
a second portion comprising a second amount of aspirin and an agent suitable for treating a cardiovascular disease or condition,
wherein the second portion is formulated to be substantially more difficult than the first portion to disintegrate or dissolve intraorally but is ingestible and releasable in the gastrointestinal track of the subject.

### BRIEF DESCRIPTION OF THE DRAWINGS

FIG. 1 illustrates the Global Flush Severity Scale used in Example 1.

FIG. 2 presents the flush rates experienced by patients as described in Example 1 and shows that patients receiving both intraorally released and gastrointestinally released aspirin had the highest anti-flush effect.

FIG. 3 presents the scale for severity of headache pain as discussed in Examples 4 and 5.

FIG. 4 presents the details of headache reduction in patients as tested and described in Example 4.

FIG. 5 presents that details of headache reduction in patients as tested and described in Example 5.

### DETAILED DESCRIPTION

The present disclosure provides pharmaceutical compositions for oral administration of aspirin and optionally with other therapeutic agents for the prevention and treatment of pain, heart diseases, cancer and other diseases. One aspect of the disclosure relates to the discovery that oral administration of aspirin achieved a remarkably higher therapeutic effect when the aspirin is partially released intraorally and partially released through the gastrointestinal (GI) track, as compared to intraoral or GI release alone.

### A. Definitions

Unless defined otherwise, the terms used herein are intended to have their ordinary meaning in the art.

All numerical designations, e.g., pH, temperature, time, concentration, and weight, including ranges, are approximations that typically may be varied (+) or (−) by increments of 0.1, 1.0, 10.0, or 100.0 as appropriate. It is to be understood, although not always explicitly stated, that all numerical designations are preceded by the term “about”.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10%, or 5%, or 2% or 1% or 0.5% of the particular term.

As used herein, the term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used in the specification and claims, the singular form “a”, “an”, and “the” includes plural references unless the context clearly dictates otherwise.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Cardiovascular disease” generally refers to conditions that involve diseases of vessels that can lead to narrowing or blockages and resulting in heart attack, chest pain (angina), congestive heart failure, arterial dissection, aneurysm, peripheral arterial disease, renal disease, stroke, and a variety of other diseases well known to those familiar to the field.

“Cardiac disease” refers to all diseases of the heart, including that that lead to narrowing or blockages and resulting in heart attack, chest pain (angina), and congestive heart failure, but also include other diseases of the heart including valvular disease, cardiomyopathy, pericarditis, myocarditis, congenital cardiac anomalies, septal defects, aneurysms, and a variety of other diseases well known to those familiar to the field.

As used herein, “compressed” dosage form (e.g., “compressed portion”), refers to a dosage form comprising a compressed powder. For example, a compressed portion may be formed using a rotary tablet press or other similar machinery known to one of skill in the art.

As used here, “disintegrates or dissolves intraorally” refers to that a majority of a composition or a portion of a composition, such as a tablet or a capsule, breaks apart into smaller particles intraorally. The majority, in one aspect, means at least about 50%, or alternatively at about 60%, or 70%, or 80%, or 90%, or 95%, or 98%, or 99%.

As used herein, “bilayer” compressed dosage form (e.g., “bilayer tablet”) refers to a single compressed dosage form comprising two layers. A bilayer compressed dosage form can be made in a single compression step. Likewise, a “trilayer” compressed dosage form (e.g., “trilayer tablet”) refers to a single compressed dosage form comprising three layers.

As used herein, “wet granulation” refers to a process known in the pharmaceutical arts that involves forming granules by the addition of a liquid, such as purified water, alcohol, or a binder solution.

“Controlled release form” refers to a formulation in which the active agent is included within a matrix, which matrix can be either insoluble, soluble, or partly soluble. Controlled release matrix formulations of the insoluble type are also referred to as insoluble polymer matrices, swellable matrices, or lipid matrices depending on the components that make up the matrix. Controlled release matrix formulations of the soluble type are also referred to as hydrophilic colloid matrices, erodible matrices, or reservoir systems. Controlled release formulations of the present disclosure refer to formulations comprising an insoluble matrix, a soluble matrix or a combination of insoluble and soluble matrices in which the rate of release is slower than that of an uncoated non-matrix or immediate release formulations or uncoated normal release matrix formulations. Controlled release formulations can be coated with a control releasing coat to further slow the release of active agent from the controlled release matrix formulation. Such coated controlled release matrix formulations can exhibit modified-release, controlled-release, sustained-release, extended-release, prolonged-release, delayed-release, or combinations thereof, of active agent.

“Controlled release coat” refers to a functional coat which can, for example, include at least one pH independent or pH dependent (such as for example enteric or reverse enteric types) polymer, soluble or insoluble polymer, lipids or lipidic materials, or combinations thereof, which, when applied onto a formulation can slow (for example, when applied to an immediate release formulation or a normal release matrix formulation), further slow (for example when applied to a controlled release matrix formulation), or modify the rate of release of an active agent.

“Excipient” refers to a pharmacologically inactive substance used with the active agents or drugs of a medication or a formulation. Excipients are also sometimes used to bulk up formulations that contain very potent active ingredients, to allow for convenient and accurate dosage. In addition to their use in the unit dose forms, excipients can be used in the manufacturing process to aid in the handling of the active substance concerned. Depending on the route of administration, and form of medication, different excipients may be used. Examples of an excipient includes, without limitation, one or more of the following: an additive, an anti-foaming agent, a binder, a chemical stabilizer, a coloring agent, a diluent, a disintegrating agent, an emulsifying agent, a filler, a flavoring agents, a glidant, a lubricant, a pH modifier, a plasticizer, a solubilizer, a swelling enhancer, a spheronization aid, a solubility enhancer, or a suspending agent.

“Immediate release formulation” refers to a formulation from which the drug is released without any substantial delay and substantially at once.

“Patient” or “subject” refers to mammals, including humans and animals, such as simians, cattle, horses, dogs, cats, and rodents having the need to take aspirin.

“Pharmaceutically acceptable salt” refers to pharmaceutically acceptable salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use, 2002, incorporated herein by reference.

“Plasticizer” refers to a compound capable of plasticizing or softening a polymer or a binder. Plasticizers can broaden the average molecular weight of a polymer in which they are included thereby lowering its glass transition temperature or softening point. Plasticizers also can reduce the viscosity of a polymer. The use of plasticizers is optional, but they can be included in a formulation to modify the properties and characteristics of the polymers used in the coat(s) or core of the formulation for convenient processing during manufacture of the coat(s) and/or the core of the formulation. Once the coat(s) and/or core has been manufactured, certain plasticizers can function to increase the hydrophilicity of the coat(s) and/or the core of the formulation in the environment of use. During manufacture of the coat(s) and/or core, the plasticizer can lower the melting temperature or glass transition temperature (softening point temperature) of the polymer or binder.

“Solid formulation” refers to a formulation that is neither liquid nor gaseous. Solid formulations include tablets, powders, microparticles, capsules, matrix forms, suppositories, sachets, troches, patches and lozenges. Solid formulations in the form of capsules contain a solid composition within a capsule that can be made of gelatin or other encapsulating material. Liquid formulations include liquid suspensions and elixirs.

“Swelling enhancer” refers to an excipient that swells rapidly resulting in an increase in the size of the tablet. At lower concentrations, these excipients can be used as super disintegrants; however at higher concentrations, e.g., at concentrations above about 5% w/w, these excipients function as swelling enhancers and increase the size of the matrix formulation.

“Therapeutically effective amount” refers to an amount of the drug that, when administered to a patient, will have the intended therapeutic effect, e.g., alleviation, amelioration, palliation or elimination of one or more manifestations of cancer or other hyperproliferative disease in the patient. A therapeutic effect does not necessarily occur by administration of one dose, and may occur only after administration of a series of doses. Typically, cancer drugs are administered in a repeating series of doses, and in certain instances each series may be referred to as a “cycle” of therapy. Thus, a therapeutically effective amount may be administered in one or more administrations.

The term “subtherapeutic amount” or “synergistically therapeutic amount” typically refers to a less than standard therapeutic amount of a drug, meaning that the amount required for the desired effect is lower than when the drug is used alone. In one aspect, the subtherapeutic amount varies depending on the desired effect. In this respect, therefore, the subtherapeutic amount of a drug for one desired effect may be actually higher than the therapeutic amount of the same drug for another desired effect. In one aspect, a subtherapeutic amount is at least about 20%, or 30%, or 40%, or 50%, or 60%, or 70, or 80%, or 90% of a therapeutically effective amount.

“Treating” or “treatment of” a condition or patient refers to taking steps to obtain beneficial or desired results, including clinical results. For purposes of this invention, beneficial or desired clinical results include, but are not limited to, in intended treatment purpose of aspirin such as reducing pain.

### B. Therapeutic Methods

The present disclosure provides methods for enhancing efficacy of aspirin in a variety of uses, e.g., preventing or treating heart attack, stroke or blood clot, for reducing pain and/or reducing aspirin-related or caused side effects. It has been discovered that oral administration of aspirin achieved a remarkably higher therapeutic effect when the aspirin is partially released intraorally and delivered transmucosally and partially released through the gastrointestinal (GI) track, as compared to intraoral or GI release alone. It is noted, in this context, that aspirin is useful for preventing or treating heart attack, stroke or blood clot formation and reducing pain.

As shown in Example 1, patients in Period IV (500 mg niacin+81 mg aspirin, half-swallowed and half-mucosally-absorbed) experienced the least severe flushing side effects, with a Global Flush Severity Scale (GFSS) average score: 3.94. This is in comparison with Period III (500 mg niacin +81 aspirin mucosally-absorbed/GFSS: 5.06) and Period II (500 mg niacin +81 aspirin swallowed/GFSS: 6.88). Further, during all periods in which aspirin was co-administered with niacin, the patients experienced less severe flush as compared to during Period I (500 mg niacin alone/GFSS: 8.44).

Additional evidence is provided in Examples 4 and 5. To summarize, we conducted two similar but distinct studies, looking at different aspirin combinations/formulations for reducing headaches. One study used a combination of aspirin, acetaminophen and caffeine. The other is simply a high-dose aspirin for headaches, in doses that are similar to those available on the market. However our studies looked at the difference in efficacy when put into our release profile, similar to the niacin/aspirin studies described in Examples 1-3.

Such data indicate that the partial intraoral release and partial GI release of aspirin achieved a synergistic effect in increasing the efficacy of aspirin across numerous and different uses and indications. It is contemplated that GI-absorbed aspirin has a different metabolic profile from intraorally absorbed aspirin which directly enters into the blood stream. This is likely due to the first-pass metabolism of the liver for medication absorbed through the GI track.

Specifically, aspirin given directly into the blood stream results in a fast peak of serum aspirin concentration, which immediately begins to drop. The immediate drop is due to the fast metabolism of aspirin into its primary metabolite, salicylic acid, resulting in high serum levels of salicylic acid. Such a quick drop, therefore, is not favorable for aspirin's anti-flush effect.

Further, salicylic acid is a reversible COX inhibitor, and is known to act competitively with aspirin itself, which is an irreversible COX inhibitor. Therefore it is further contemplated that a directly-absorbed aspirin, like those IV or intraorally absorbed, not only leads to a time limited anti-flush effect, but actually hinders the anti-flush effect of aspirin.

GI-absorbed aspirin follows a different metabolic profile, with aspirin serum levels not peaking until approximately 15-20 minutes following administration. GI-absorbed aspirin is metabolized much more slowly than aspirin given directly into the blood stream. This allows for a longer time for aspirin's irreversible COX inhibitory effect to act upon the COX receptors, in the platelets as well as the vasculature.

Directly-absorbed (such as oral mucosally absorbed) aspirin has not yet been used to enhance the therapeutic effect of aspirin. The present disclosure, however, reveals that there was a more pronounced therapeutic effect when aspirin is partially absorbed through the oral mucosa (i.e. more directly into the blood stream without first-pass liver metabolism) and partially absorbed through the GI (i.e. undergoing first-pass liver metabolism).

Accordingly, these data suggest that aspirin absorbed directly into the blood stream hits a peak serum concentration relatively quickly, within minutes. Aspirin given concurrently through the GI, on the other hand, has a serum peak concentration about 15-20 minutes later. As the former metabolizes much quicker than the latter, there then is a point after ingestion—around 30-45 minutes, when the serum aspirin given by the former means is less than that given by the latter.

Therefore, when just focusing on the aspirin serum levels, a combination of direct-absorption and GI-absorption will “smooth out” the aspirin serum concentration over time: the direct absorption getting serum levels high early, and the GI absorption keeping serums level high later. This higher and wider serum level of aspirin result in a more pronounced therapeutic effect.

The data also suggest that elimination of salicylic acid from the serum is rather quick, with most of the elimination occurring within 20 minutes. Therefore, the peak concentration from GI-absorbed aspirin is delayed, relative to the oral mucosal-absorbed aspirin. Accordingly, there is less competitive inhibition by the former's salicylic acid metabolite than what would have been expected. This is more likely in the circumstance when the drugs were given to a patient after the patient recently had a meal.

It is also contemplated that a portion of GI absorbed aspirin turns into salicylic acid before the portion that remains as aspirin has time to block the vasculature COX, therefore partially competitively inhibiting itself more than it would for the platelet COX. An earlier, directly-absorbed aspirin bolus, in this respect, also has an added therapeutic effect through vasculature COX inhibition.

The data presented in the present disclosure also show that, by partially releasing aspirin intraorally and partially releasing aspirin through the GI, the combined effect is higher than what is achieved by separate administration due to the smoothened and broadened plasma exposure profile. Accordingly, such combined dosing also give rise to reduced side effects that would normally have occurred with either intraoral or GI formulation alone. Thus, one embodiment of the present disclosure provides methods of reducing aspirin-induced side effects, such as GI ulcer.

In one embodiment, provided is a method for preventing or treating heart attack, stroke or blood clot in a subject in need thereof, comprising orally administering to the subject a first composition comprising a first amount of aspirin, and a second composition comprising a second amount of aspirin, wherein the first composition is formulated so as to, upon administration, disintegrate or dissolve intraorally providing rapid release of the aspirin of the first composition in the subject, and wherein the second composition is formulated to be substantially more difficult than the first composition to disintegrate or dissolve intraorally but is ingestible and releasable in the gastrointestinal track of the subject.

The first and second compositions can be administered concurrently or sequentially. When administered sequentially, the first composition can be administered prior to that of the second administration. In some aspects, the first and second compositions are combined into a single dosage form for concurrently administration.

In some aspects, the subject is further administered an effective amount of an agent suitable for treating a cardiovascular disease or condition. Agents suitable for treating a cardiovascular disease or condition are known in the art, including, for instance, anti-coagulants, anti-platelet agents, hypertensive medications, diabetes medications, beta blockers, ACE inhibitors, statins, aldosterones, calcium channel blockers, metformin, sulfonylurea, DPP4 inhibitors, fibrates, an anticoagulant, and eztimibe. Other non-limiting examples include dipyridamole, pravastatin, metoprolol, carvedilol, captopril, zofenopril, enalapril, ramipril, quinapril, perindopril, lisinopril, benazepril, imidapril, trandolapril, fosinopril, eplerenone, warfarin, acenocoumarol, atromentin, brodifacoum, phenindone, low molecular weight heparin, fondaparinux, idraparinux, rivaroxaban, apixaban, dabigatran, hirudin, lepirudin, and bivalirudin. In one embodiment, the dose amount of the second agent can be readily ascertained by one of skill in the art based on the patient's condition, body weight, etc. In some embodiments, due to the increased efficacy of the aspirin, the second agent may be dosed at a subtherapeutic amount.

Likewise, in another embodiment, provided is a method for reducing pain in a subject in need thereof, comprising orally administering to the subject a first composition comprising a first amount of aspirin, and a second composition comprising a second amount of aspirin, wherein the first composition is formulated so as to, upon administration, disintegrate or dissolve intraorally providing rapid release of the aspirin of the first composition in the subject, and wherein the second composition is formulated to be substantially more difficult than the first composition to disintegrate or dissolve intraorally but is ingestible and releasable in the gastrointestinal track of the subject.

The first and second compositions can be administered concurrently or sequentially. When administered sequentially, the first composition can be administered prior to that of the second administration. In some aspects, the first and second compositions are combined into a single dosage form for concurrent administration.

In some aspects, the subject is further administered an effective amount of a painkiller. Painkillers are known in the art, including, for instance, a non-steroidal anti-inflammatory drug (NSAID), a COX-2 inhibitor, an opioid, an anxiolytic, a muscle relaxant, a methylxanthine (such as caffeine), a salicylate, magnesium, tripatans, ergots, an anti-nausea agent (e.g., reglan, zofran, compazine, phenergan), an anti-depressant, a selective serotonin reuptake inhibitor (SSRI). Other more specific examples include acetaminophen, caffeine, butalbital, codeine, hydrocodone, oxycodone, hydromorphone, oxymorphone, pentazocine, dextropropoxyphene, propoxyphene, amitriptyline, carbamazepine, gabapentin, pregabalin and flupirtine. In one embodiment, the aspirin is combined with acetaminophen and caffeine. In one embodiment, the dose amount of the second agent can be readily ascertained by one of skill in the art based on the patient's condition, body weight, etc. In some embodiments, due to the increased efficacy of the aspirin, the second agent may be dosed at a subtherapeutic amount. In some embodiments, due to the increased efficacy of the aspirin, the aspirin may be dosed at what would normally be considered a subtherapeutic amount.

For any of the above embodiments, in some aspects, the first composition disintegrates or dissolves intraorally within about 10 minutes. In other aspects, the first composition disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds, or about 2 seconds, or less than 1 second. In another aspect, the first composition is absorbed transmucosally in the oral cavity within about 10 minutes, or about 9, or about 8, or about 7, or about 6, or about 5, or about 4, or about 3 or about 2 minutes, or alternatively about 60 seconds, or about 50, or about 40, or about 30, or about 20, or about 10, or about 5 seconds, or about 2 seconds, or less than 1 second.

In one aspect, the aspirin in the first composition is a subtherapeutic amount, such as but not limited to, from about 10 mg to about 1000 mg. In one aspect, the aspirin in the first composition is at least about 10 mg, or at least about 20 mg, 30 mg, 40 mg, 50 mg, or 100 mg. In another aspect, the aspirin in the first composition is no more than about 150 mg, 200 mg, 250 mg, 300 mg, 325 mg, 400 mg, 500 mg, 600 mg or 650 mg, or 1000 mg. In one aspect, the aspirin in the second composition is a subtherapeutic amount, such as but not limited to, from about 10 mg to about 1000 mg. In one aspect, the aspirin in the second composition is at least about 10 mg, or least about 20 mg, 30 mg, 40 mg, 50 mg, or 100 mg. In another aspect, the aspirin in the second composition is no more than about 150 mg, 200 mg, 250 mg, 300 mg, 325 mg, 400 mg, 500 mg, 600 mg or 650 mg, or 1000 mg. In another aspect, the aspirin in both the first and second compositions is a subtherapeutic amount, such as but not limited to, from about 10 mg to about 1000 mg, or is at least about 10 mg, or least about 20 mg, 30 mg, 40 mg, 50 mg, or 100 mg, or is no more than about 150 mg, 200 mg, 250 mg, 300 mg, 325 mg, 400 mg, 500 mg, 600 mg or 650 mg.

In one aspect, the aspirin in the first composition is at least about 10%, or 20%, or 30%, or 40%, of 50%, or 60%, or 70%, or 80%, or 90% of a therapeutically effective amount. In one aspect, the aspirin in the first composition is at most about 10%, or 20%, or 30%, or 40%, of 50%, or 60%, or 70%, or 80%, or 80% of a therapeutically effective amount. In one aspect, the aspirin in the second composition is at least about 10%, or 20%, or 30%, or 40%, of 50%, or 60%, or 70%, or 80%, or 80% of a therapeutically effective amount. In one aspect, the aspirin of in the second composition is at most about 10%, or 20%, or 30%, or 40%, of 50%, or 60%, or 70%, or 80%, or 80% of a therapeutically effective amount.

In one aspect, the aspirin in the first composition constitutes at least about 10% of the total aspirin. Alternatively, the aspirin in the first composition constitutes at least about 20%, or 30%, or 40%, or 50%, or 60%, or 70%, or 80%, or 90% of the total aspirin. In some aspects, however, the aspirin in the first composition can be less than about 20%, or 30%, or 40%, or 50%, or 60%, or 70%, or 80%, or 90% of the total aspirin. In a particular aspect, the aspirin in the first composition constitutes from about 40% to about 60%, or alternatively from about 45% to about 55% of the total aspirin.

In one aspect, the total amount of aspirin in the composition is greater than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, or 200 mg. In another aspect, the total amount of aspirin in the composition is less than about 50, mg, 75 mg, 100 mg, 110 mg, 120 mg, 130 mg, 140 mg, 150 mg, 160 mg, or 165 mg, or 170 mg, or 180 mg, or 190 mg, or 200 mg, or 250 mg, or 300 mg, or 400 mg, or 500 mg, or 600 mg, or 700 mg, or 800 mg, or 900 mg, or 1000 mg.

In yet another aspect, the therapeutically effective amount of the agent suitable for treating a cardiovascular disease or condition or the painkiller can be determined with information or methods known in the art.

In one aspect, the agent or painkiller is administered following the administration of the first and second compositions. In another aspect, the agent or painkiller is administered concurrently with the first and second compositions. In another aspect, the first and second compositions are in a single dosage form as disclosed in the disclosure. In another aspect, the agent or painkiller is also in the same dosage form as the first and second compositions. In some embodiments, the administration is with 30 minutes following a meal. In another aspect, the administration is accompanied by oral administration of an acidic drink which can assist transmucosal absorption of the first aspirin composition.

Also provided, therefore, is a method of administering aspirin to a subject with reduced side effects, comprising administering to the subject a first composition comprising a first subtherapeutic amount of aspirin and a second composition comprising a second subtherapeutic amount of aspirin, wherein the first composition disintegrates or dissolves intraorally within 10 minutes permitting rapid release of the aspirin of the first portion, and the second composition is ingested and released in the gastrointestinal track of the subject.

It is also contemplated that a single composition of aspirin can also be used to achieve the desired effect, when a portion of the aspirin is dissolved intraorally and the remaining is released in the GI track.

Thus, also provided is a method of administering aspirin to a subject with reduced side effects, comprising administering to the subject a therapeutically effective amount of aspirin, wherein a portion of the aspirin disintegrates or dissolves intraorally within 10 minutes permitting rapid release of the aspirin in the portion, and the remaining aspirin is ingested and released in the gastrointestinal track of the subject.

In other aspects, the first composition or portion of the aspirin disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds.

### C. Oral Dosage Forms

One embodiment of the present disclosure provides a pharmaceutical formulation comprising a first portion comprising a first amount of aspirin formulated so as to, upon administration to a subject, disintegrate or dissolve intraorally; and a second portion comprising a second amount of aspirin and an agent suitable for treating a cardiovascular disease or condition, wherein the second portion is formulated to be substantially more difficult than the first portion to disintegrate or dissolve intraorally but is ingestible and releasable in the gastrointestinal track of the subject.

Agents suitable for treating a cardiovascular disease or condition are known in the art, including, for instance, anti-coagulants, anti-platelet agents, hypertensive medications, diabetes medications, beta blockers, ACE inhibitors, statins, aldosterones, calcium channel blockers, metformin, sulfonylurea, DPP4 inhibitors, fibrates, anticoagulants, and eztimibe. Other non-limiting examples include dipyridamole, pravastatin, metoprolol, carvedilol, captopril, zofenopril, enalapril, ramipril, quinapril, perindopril, lisinopril, benazepril, imidapril, trandolapril, fosinopril, eplerenone, warfarin, acenocoumarol, atromentin, brodifacoum, phenindone, low molecular weight heparin, fondaparinux, idraparinux, rivaroxaban, apixaban, dabigatran, hirudin, lepirudin, and bivalirudin.

One embodiment of the present disclosure provides a pharmaceutical formulation comprising a first portion comprising a first amount of aspirin formulated so as to, upon administration to a subject, disintegrate or dissolve intraorally; and a second portion comprising a second amount of aspirin and a painkiller, wherein the second portion is formulated to be substantially more difficult than the first portion to disintegrate or dissolve intraorally but is ingestible and releasable in the gastrointestinal track of the subject.

Painkillers are known in the art, including, for instance, a non-steroidal anti-inflammatory drug (NSAID), a COX-2 inhibitor, an opioid, an anxiolytic, a muscle relaxant, a methylxanthine (such as caffeine), a salicylate, magnesium, tripatans, ergots, an anti-nausea agent (e.g., reglan, zofran, compazine, phenergan), an anti-depressant, a selective serotonin reuptake inhibitor (SSRI). Other more specific examples include acetaminophen, caffeine, butalbital, codeine, hydrocodone, oxycodone, pentazocine, dextropropoxyphene, propoxyphene, amitriptyline, carbamazepine, gabapentin, pregabalin and flupirtine. In one embodiment, the composition comprises aspirin as described herein, acetaminophen, and caffeine.

In some aspects, the pharmaceutical formulation is in the form of a tablet or a capsule. In one aspect, the pharmaceutical formulation is a tablet and the first portion and second portion constitute separate layers. In one aspect, the layer of the first portion is adjacent to the layer of the second portion. In one aspect, the layer of the first portion surrounds or encloses the layer of the second portion. In the formulation, the agent suitable for treating a cardiovascular disease or condition or the painkiller forms a separate portion from the first and second portions, or alternatively can be part of the first or second portion.

In one aspect, the aspirin of the first portion is at a subtherapeutic amount, such as but not limited to, from about 10 mg to about 1000 mg. In one aspect, the amount of aspirin of the first portion is at least about 10 mg, or least about 20 mg, 30 mg, 40 mg, 50 mg, or 100 mg. In another aspect, the amount of aspirin of the first portion is no more than about 150 mg, 200 mg, 250 mg, 300 mg, 325 mg, 400 mg, 500 mg, 600 mg or 650 mg, or 1000 mg. In one aspect, the aspirin in the second portion is at a subtherapeutic amount, such as but not limited to, from about 10 mg to about 1000 mg. In one aspect, the amount of aspirin in the second portion is at least about 10 mg, or least about 20 mg, 30 mg, 40 mg, 50 mg, or 100 mg. In another aspect, the amount of aspirin in the second portion is no more than about 150 mg, 200 mg, 250 mg, 300 mg, 325 mg, 400 mg, 500 mg, 600 mg or 650 mg, or 1000 mg.

In one aspect, the aspirin of the first portion is at least about 10%, or 20%, or 30%, or 40%, of 50%, or 60%, or 70%, or 80%, or 90% of a therapeutically effective amount. In one aspect, the aspirin of the first portion is at most about 10%, or 20%, or 30%, or 40%, of 50%, or 60%, or 70%, or 80%, or 80% of a therapeutically effective amount. In one aspect, the aspirin of the second portion is at least about 10%, or 20%, or 30%, or 40%, of 50%, or 60%, or 70%, or 80%, or 80% of a therapeutically effective amount. In one aspect, the aspirin of the second portion is at most about 10%, or 20%, or 30%, or 40%, of 50%, or 60%, or 70%, or 80%, or 80% of a therapeutically effective amount.

In one aspect, the first portion of aspirin constitutes at least about 10% of the total aspirin. Alternatively, the first portion of aspirin constitutes at least about 20%, or 30%, or 40%, or 50%, or 60%, or 70%, or 80%, or 90% of the total aspirin. In some aspects, however, the first portion of aspirin can be less than about 20%, or 30%, or 40%, or 50%, or 60%, or 70%, or 80%, or 90% of the total aspirin. In a particular aspect, the first portion constitutes from about 40% to about 60%, or alternatively from about 45% to about 55% of the total aspirin.

In one aspect, the total amount of aspirin in the formulation is greater than about 50 mg, or 60 mg, or 70 mg, or 80 mg, or 90 mg, or 100 mg, or 120 mg, or 140 mg, or 150 mg, or 160 mg, or 165 mg, or 170 mg, or 180 mg, or 190 mg, or 200 mg. In another aspect, the total amount of aspirin in the composition is less than about 50 mg, 75 mg, 100 mg, 110 mg, 120 mg, 130 mg, 140 mg, 150 mg, 160 mg, or 165 mg, or 170 mg, or 180 mg, or 190 mg, or 200 mg, or 250 mg, or 300 mg, or 400 mg, or 500 mg, or 600 mg, or 700 mg, or 800 mg, or 900 mg, or 1000 mg.

In yet another aspect, the therapeutically effective amount of the agent suitable for treating a cardiovascular disease or condition or the painkiller can be determined with information or methods known in the art.

The compositions of the present disclosure are clearly distinguishable from what has been disclosed in the prior art, in which only one type of aspirin formulation is used, see, e.g., U.S. Pat. No. 8,404,275. On the other hand, the disclosure of U.S. Pat. No. 8,404,275 is incorporated as reference into the present disclosure to more fully describe compositions and methods suitable for preparing the compositions of the present disclosure.

Another aspect of the invention provides a process of preparing the disclosed compositions. In some embodiments, the process comprises forming a first portion and a second portion and compressing the first and second portions to form a bilayer or two-halves compressed solid oral dosage form. Preparation of each portion is further described below.

### 1. First Portion of Aspirin for Intraoral Release

Methods of preparing a composition suitable for intraoral release are known in the art. In one aspect, the first portion further includes a film-coating agent, an excipient, a binder, a lubricant, or a plasticizer.

In one aspect, the first portion disintegrates or dissolves intraorally within about 10 minutes. In other aspects, the first portion disintegrates or dissolves intraorally within about 9 minutes, or about 8, or about 7, or about 6, or about 5, or about 4, or about 3 or about 2 minutes, or alternatively about 60 seconds, or about 50, or about 40, or about 30, or about 20, or about 10, or about 5 seconds, or about 2 seconds, or less than 1 second.

In some aspects, the first portion is chewable. In some aspects, the first portion is in the form of molded triturate.

In one aspect, the first portion further includes an agent that promotes the oral or buccal absorption of aspirin. Non-limiting examples of such agents include bile acid salts, sodium lauryl sulfate, lysalbinic acid, salicylic acid, 5-methoxy salicylic acid, 3,4-dihydroxy phenyl acetic acid (DOPAC) and homovanillic acid and their sodium salts thereof. Other hydroxyaryl acids, such as 1-hydroxy-2-naphthoic acid, naphthoresorcyclic acid, ferulic acid, caffeic acid, resorcylic acid and gentisic acid, have similar effects.

The amount of hydroxyaryl or hydroxyaralkyl acid or salt, amide or ester derivatives thereof forms may vary over a wide range; in general, the identity and the amount of the hydroxyaryl or hydroxyaralkyl acids or salt, amide or ester thereof is used in connection with the drug in order to be effective in enhancing the absorption rate of the drug into the bloodstream.

In another aspect, the first portion further includes a disintegrant. Non-limiting examples of disintegrants include crospovidone, crystalline cellulose, hydroxypropylcellulose with a low degree of substitution, croscarmellose sodium, carmellose calcium, carboxystarch sodium, carboxymethyl starch sodium, potato starch, wheat starch, corn starch, rice starch, partly pregelatinized starch, and hydroxypropyl starch. One or two or more of these can be used together. Coating with a disintegrant also contributes to improvement of compression moldability.

### 2. Second Portion of Aspirin and Optionally a Painkiller or Cardiovascular Medication

The second and third portions of the composition can be prepared with methods known in the art for a typical oral dosage form suitable for GI absorption. Like the first portion, the second portion can also include a film-coating agent, an excipient, a binder, a lubricant, or a plasticizer.

Compared to the first portion, the second is substantially more difficult to disintegrate or dissolve intraorally. This can be achieved chemically or physically. For instance, the second portion can be physically harder. In one aspect, the second portion is compressed. In another aspect, the second portion has a hardness that is at least about 10 kilopascal (kp), or alternatively about 11, or 12, or 13, or 14, or 15, or 20, or 25 or 30 or 40 or 50 kp.

Hardness can be assessed by means commonly used in the art, for example, using commercially available hardness testers that are routinely used for assessing the hardness of pharmaceutical dosage forms.

In some aspects, the second portion further comprises a pharmaceutically acceptable flavoring agent not present in the first portion. The flavoring agent provides a flavor that alerts the patients that this portion should not be chewed and needs to be swallowed so as to increase patient compliance.

In one aspect, the aspirin in the second portion constitutes at least about 10% of the total aspirin. Alternatively, the aspirin in the second portion constitutes at least about 20%, or 30%, or 40%, or 50%, or 60%, or 70%, or 80%, or 90% of the total aspirin. In some aspects, however, the aspirin in the second portion can be less than about 20%, or 30%, or 40%, or 50%, or 60%, or 70%, or 80%, or 90% of the total aspirin. In a particular aspect, the aspirin in second portion constitutes from about 40% to about 60%, or alternatively from about 45% to about 55% of the total aspirin. In one aspect, the ratio of aspirin between the first portion and the second portion is about 1:1. Alternatively, the ratio is at least about 1:4, or 1:3, or 1:2 or 1:1.5, or is no more than about 4:1, 3:1, 2:1 or 1.5:1.

The pharmaceutical composition of the present disclosure can be in the form of a tablet or capsule. When in the form of a tablet, the second portion, in one aspect, is enclosed within the first portion or alternatively partially exposed.

When the composition is in the form of a tablet, the tablet can include an outer portion and an inter portion, with the outer portion containing the first portion and the inner portion containing the second portion and optionally the third portion.

In one aspect, the outer portion is formulated to dissolve in the oral cavity of a subject and to release the aspirin in the first portion across the oral mucosa of the subject. In one aspect, the inner portion is harder than the outer portion and is formulated for dissolving in stomach, intestines, or further distal in the gastrointestinal tract of the subject.

In one aspect, the inner portion comprises a texture on the surface that is recognizable by the tongue of a subject. In another aspect, the outer portion comprises a water soluble sugar or sugar substitute. In another aspect, the outer portion is surrounded by a thin shell to allow encapsulation of liquid, powder or gel in the outer portion.

In one aspect, the outer potion is flavored or sweetened. In one aspect, the tablet further comprises an intermediate layer between the outer and inner portions. In one aspect, the intermediate layer comprises enteric coating. In one aspect, the inner portion is formulated to absorb a biting shock and not break a tooth. In another aspect, the tablet comprises a layer of aspirin which breaks down in the mouth, but this layer has particles within it that don't completely break down in the mouth and stay full particles, such that there is partial intraoral release and, when the particles as swallowed, partial gastrointestinal release.

The pharmaceutical composition of the above embodiments can further include a third portion that comprises an effective amount of a painkiller or a cardiovascular medication, that is, an agent suitable for treating a cardiovascular disease or condition. In one aspect, the third portion is in the form of controlled release. In another aspect, the third portion further comprises enteric coating. In yet another aspect, the third portion is enclosed in the first portion or the second portion.

### 3. Additional Additives to the Composition

In yet another aspect, either or both of the first portion and second portion further comprises excipients, lubricants, pH adjusters, taste-masking agents, sweeteners, acidifiers, refrigerants, foaming agents, preservatives, fluidizers, antioxidants, colorants, stabilizers, surfactants, buffering agents, flavors, binders or drug solubilizers. A person skilled in the art may immediately list specific examples of these additives.

Any excipient used for pharmaceutical preparations can be used without limitation, but examples of excipients used in the tablet of the present invention can include sugars such as erythritol, mannitol, xylitol, sorbitol, lactitol, paratinit, paratinose, maltitol, maltose, trehalose, lactose, sucrose, glucose, olygosaccharides, fructose and maltose and the like. One or two or more kinds of these excipients can be used.

Various embodiments of the composition may include pharmaceutically acceptable binders (adhesives). Binders are agents that impart cohesive properties to powdered materials through particle-particle bonding. Examples of suitable binders include celluloses and crosslinked polyvinyl pyrrolidone, matrix binders (dry starch, dry sugars), film binders (polyvinyl pyrrolidone (PVP), starch paste, celluloses, bentonite, sucrose), and chemical binders (polymeric cellulose derivatives, such as carboxy methyl cellulose, hydroxypropylcellulose (HPC) and hydroxypropylmethylcellulose (HPMC); sugar syrups; corn syrup; water soluble polysaccharides such as acacia, tragacanth, guar and alginates; gelatin; gelatin hydrolysate; agar; sucrose; dextrose; and non-cellulosic binders, such as polyvinyl pyrrolidone, polyethylene glycol (PEG), vinyl pyrrolidone copolymers, pregelatinized starch, sorbitol, glucose, microcrystalline cellulose, such as FMC BioPolymer's Avicel® PH101 and Avicel® PH102, and silicified microcrystalline cellulose, such as Penwest Pharmaceutical's ProSolv SMCC™). In specific embodiments, a binder is selected from the group consisting of corn starch, potato starch, polyvinyl pyrrolidone, hydroxypropylmethyl cellulose, and hydroxylpropyl cellulose. A binder may be included in any portion of the dosage form, such as the intragranular portion and/or extragranular portion of either or both of the first and second layers.

In some embodiments, the composition further comprises a pharmaceutically acceptable diluent or filler. Pharmaceutically acceptable diluents include, but are not limited to, lactose (such as lactose monohydrate, lactose anhydrous, and DMV International's Pharmatose® DCL21 crystalline alpha monohydrate milled lactose), mannitol, talc, magnesium stearate, sodium chloride, potassium chloride, citric acid, spray-dried lactose, starch, hydrolyzed starches, directly compressible starch, microcrystalline cellulose (such as Avicel® PH101 and Avicel® PH102), cellulosics, sorbitol, sucrose, glucose, sucrose-based materials, saccharides, calcium sulfate, dibasic calcium phosphate (such as Emcompress®) and dextrose, and/or mixtures of any of the foregoing. In specific embodiments, a diluent is selected from the group consisting of microcrystalline cellulose, lactose, mannitol, dicalcium phosphate, dextrose, compressible sugar, and spray-dried lactose with microcrystalline cellulose. A diluent may be may be included in any portion of the dosage form, such as the intragranular portion and/or extragranular portion of either or both of the first and second layers.

In some embodiments, the composition comprises magnesium stearate. In specific embodiments, the magnesium stearate is present in a range of about 0.5% to 2% w/w, based on the total weight of the layer.

In some embodiments, the diluent is microcrystalline cellulose or microlac (spray-dried lactose with microcrystalline cellulose). In specific embodiments, the microcrystalline cellulose or microlac is present in a range of about 20% to 60% w/w, based on the total weight of the layer.

Various embodiments of the invention may include pharmaceutically acceptable anti-adherents (anti-sticking agents, glidants, flow promoters, lubricants) such as talc, colloidal silicon dioxide, such as Aerosil® 200, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, calcium stearate, silica gel, starch, hydrogenated vegetable oils, sodium benzoate, sodium acetate, leucine, PEG-4000, and magnesium lauryl sulfate. In specific embodiments, an anti-adherents is selected from glidants and lubricants. Suitable glidants include, but are not limited to, colloidal silicon dioxide (Aerosil®), magnesium trisilicate, talc, and tribasic calcium phosphate. Suitable lubricants include, but are not limited to magnesium, aluminum, calcium, zinc stearate, and talc. An anti-adherent may be included in any portion of the dosage form, such as the intragranular portion and/or extragranular portion of either or both of the first and second layers. In specific embodiments, an anti-adherent is included in the extragranular portion of the first layer and/or the extragranular portion of the second layer.

In some embodiments, the glidant is talc. In specific embodiments, talc is present in a range of about 1% to 7% w/w, based on the total weight of each layer.

### EXAMPLE 1

This example demonstrates the anti-flush effect of concurrent administration of both intraorally released and gastrointestinal tract (GI) released aspirin.

Healthy human patients were recruited for this study. Each patient did not have an allergy or reaction to aspirin or niacin, had not been diagnosed with kidney disease or liver disease, were not pregnant or planning to be pregnant within the following two months, had not been breastfeeding within the preceding two months, and had not used aspirin for the preceding 7 days.

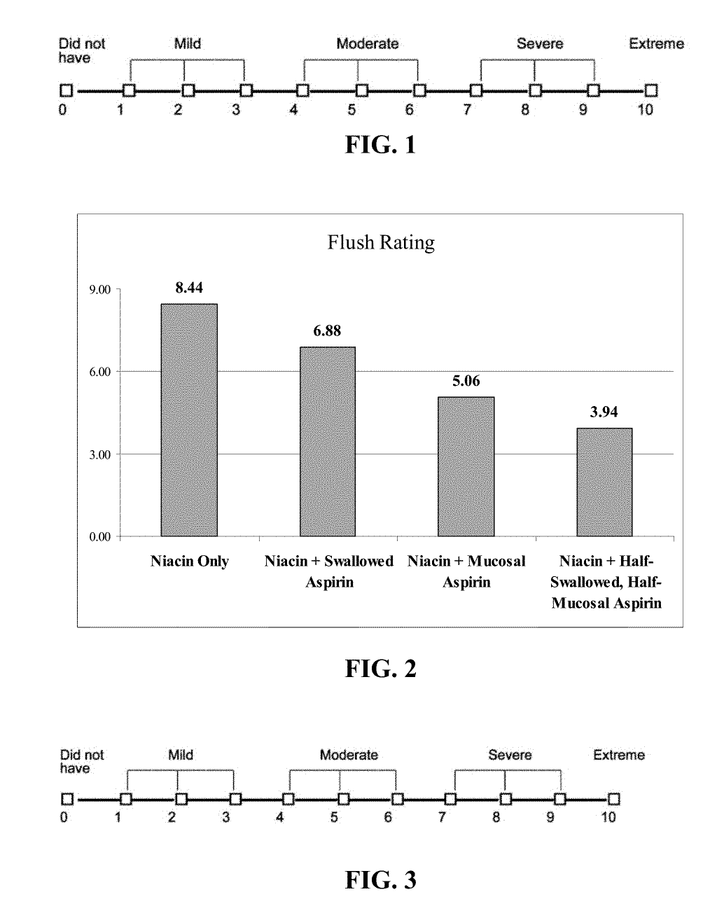

In Period I, each patient was given 500 mg niacin orally. Each patient was asked to rate his or her flush on the Global Flush Severity Scale (GFSS) (see FIG. 1 and Paolini et al. Int. J. Clin. Pract. 62(6):896-904 (2008)), when the flush completely resolved. The Global Flushing Severity Score measures, overall, in the previous 24 hours, how each patient rates the flushing symptoms, including redness, warmth, tingling, and itchiness of the skin.

Period II did not start until at least two days upon completion of Period I. At Period II, each patient orally swallowed 81 mg aspirin followed by 500 mg niacin. After the flush completely resolved, then each patient recorded his or her GFSS flush rating.

Not until at least two days later did Period III start. At Period III, each patient was asked to not swallow the orally administered aspirin (81 mg) but to allow the aspirin to be absorbed through the oral mucosa. The aspirin was in powdered form and the remaining aspirin in the mouth was washed out with water. Afterwards, 500 mg of niacin was swallowed with a glass of water. Still, the flush was rated (GFSS) after it was resolved.

Still, at least another two days later, at Period IV, the patients were instructed to take mucosally-absorbable aspirin (81 mg) into the mouth, and chew partially and rub into the gums, lips and inside of mouth until about half dissolved (about 10 seconds), then swallow the rest with a glass of water. Then, each patient swallowed a dose of niacin (500 mg) with a glass of water. The flush was then rated (GFSS) after it completely resolved.

As shown in FIG. 2, the patients during Period IV suffered the least severe flush (3.94, a 53% reduction from Period I) than during any other Period. Among Periods I through III, the severity of flush was the lowest in Period III (5.06, a 40% reduction from Period I), second lowest in Period II (6.88, a 18% reduction from Period I) and the highest in Period I (8.44). As the total amount of aspirin was the same among Period II-IV, this example therefore demonstrates the synergistic effect between intraorally released aspirin and GI-released aspirin.

### EXAMPLE 2

This example assesses the level of flushes caused by several combinations of niacin and aspirin.

### Study Design

After administration of a screening questionnaire, this study will enroll healthy volunteers meeting inclusion criteria into a cross-over study designed to measure the GF SS at specific time intervals after ingestion of the following regimens.

Standardizing Dose: [Niacin 1000 mg].
Group A: [Chewed Aspirin 162 mg]+[Swallowed Placebo 162 mg]+[Niacin 1000 mg]
Group B: [Chewed Placebo 162 mg]+[Swallowed Aspirin 162 mg]+[Niacin 1000 mg]
Group C: [Chewed Aspirin 81 mg+Chewed Placebo 81 mg]+[Swallowed Aspirin 81 mg+Swallowed Placebo 81 mg]+[Niacin 1000 mg]

Subjects will each first be given a single, immediate-release dose of 1000 mg of niacin alone, (a “calibration dose”) and asked to rate the resultant flush, in order to give them the sense for how strongly the niacin flush is as a basis for comparison.

Then, after at least 24 hours (a “washout period”) have passed, subjects will be given the above dosing regimens in a double-blind fashion, and asked to rate their flush at 30-minute intervals using a modified version of the GFSS (a 0-10 rating score) until the flush is completely reduced, or for three hours. Soda will be used for the chewable pills to improve mucosal absorption. Dosing of the medications will be at least 2 days apart, to allow for aspirin elimination. The study will take approximately 3 hours per dosing, with 5 total doses spaced at least two days apart, to allow for a wash out period. The total study duration will be one-two weeks.

Inclusion criteria: Adults, age 18 or older.

Exclusion criteria: known allergy to aspirin, niacin or willow bark; known renal disease; known liver disease; known pregnancy; breast-feeding; and use of aspirin in the last 7 days.

Materials: All subjects will receive the same over-the-counter medications, such as Niacin (B3) 1000 mg capsules (Twinlab), and aspirin (Bayer chewable low dose 81 mg;). A placebo similar to 81 mg aspirin will also be given,

Statistical Analysis: Powering to detect an approximate 10% reduction in flush will require approximately 22 subjects. Assuming a 40% drop out rate, we will target the recruitment of 40 subjects.

Using the modified GFSS, each subject reports a flushing score from 1 to 10 (none 0, mild 1 to 3, moderate 4 to 6, severe 7 to 9, extreme 10), at 30 minute intervals, for the duration of the flushing sensation, up to 3 hours. The study will calculate a sum total flush score for each patient on each dosing regimen, as well as record each significant flushing event (a score of 4 or higher). The length of flush duration will also be recorded. These data will be compared between each regimen in each individual subject, and also compared amongst all subjects.

Primary Endpoint: Reduction in total flush score when compared with niacin alone.

Secondary Endpoints: Reduction in number of significant flushing events, and as a representation of the duration of the flush.

It is contemplated that Group C that includes both chewed and swallowed aspirin leads to the most flush reduction for niacin.

### EXAMPLE 3

This example is similar to Example 2, but uses different amounts of aspirin and niacin. In this example, each dosing regimen has a total of 203 mg aspirin. One dose has 203 mg of chewable aspirin, and a swallowed placebo, and 500 mg niacin. Another dosing has 203 mg of swallowed aspirin, and chewable placebo, and 500 mg niacin. A third dosing has 122 mg of chewable aspirin, 81 mg of swallowed aspirin, and 500 mg niacin. The niacin in each dosing period is 500 mg. In addition to being used with the chewable pills, soda can also be used for the washout.

Additionally, different from Example 2, the niacin pills in this example have an enteric coating to further delay the release. It is noted that the enteric coating delays the release, rather than extends the release duration. It is contemplated that the dosing regimen that includes both chewed and swallowed aspirin will cause the most flush reduction for niacin.

### EXAMPLE 4

This example demonstrates the anti-headache effect of both intraorally released and gastrointestinal tract (GI) released aspirin, when combined with a GI released acetaminophen and caffeine.

Healthy human patients who experience non-specific headaches were recruited for this study. Each patient did not have an allergy or reaction to aspirin, caffeine, or acetaminophen, had not been diagnosed with kidney disease or liver disease, were not pregnant or planning to be pregnant within the following two months, had not been breastfeeding within the preceding two months, and had not used aspirin for the preceding 7 days.

In Period I, patients waited until they had a headache. Each patient was asked to rate his or her headache on the 10-point scale (see FIG. 3). The patient then swallowed 486 mg of aspirin (6 tablets of 81 mg each) without chewing, immediately followed by swallowing a combination of pill 1000 mg acetaminophen and 130 mg caffeine. Each patient was asked to rate his or headache again on the 10-point scale 30-minutes later.

Period II did not start until at least two days upon completion of Period I. At Period II, patients first waited until they had a headache, each patient was asked to rate his or her headache on the 10-point scale. The patient then chewed 486 mg of aspirin (6 tablets of 81 mg each) and rubbed the aspirin in their oral mucosa, lips, gums and mouth and was asked to be careful not to swallow any of the aspirin, but to wash out their mouth afterwards with a sip of soda and spitting out such that no aspirin was swallowed. This was immediately followed by the patient swallowing a combination pill of 1000 mg acetaminophen and 130 mg caffeine. Each patient was asked to rate his or headache again on the 10-point scale 30-minutes later.

Not until at least two days later did Period III start. At Period III, patients first waited until they had a headache, and each patient was asked to rate his or her headache on the 10-point scale. The patient then chewed 486 mg of aspirin (6 tablets of 81 mg each) and rubbed the aspirin in their oral mucosa, lips, gums and mouth for about 10 seconds or until half was dissolved, then to take a sip of soda to wash the rest, but this time to swallow the soda and aspirin such that half was swallowed. This was immediately followed by the patient swallowing a combination pill of 1000 mg acetaminophen and 130 mg caffeine. Each patient was asked to rate his or headache again on the 10-point scale 30-minutes later.

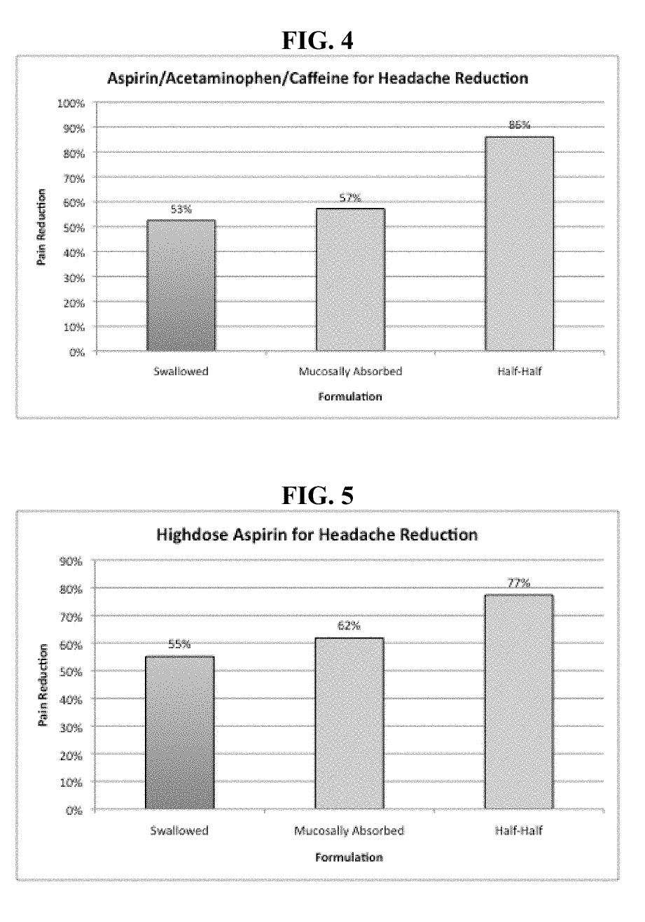

As shown in FIG. 4, patients had the largest decrease during Period III had the largest decrease in headache (77%), followed by Period II (62%) and then by Period I (55%). As the total amount of aspirin was the same among Period II-IV, this example therefore demonstrates the synergistic effect between intraorally released aspirin and GI-released aspirin.

### EXAMPLE 5

This example demonstrates the anti-headache effect of both intraorally released and gastrointestinal tract (GI) released aspirin.

Healthy human patients who experience non-specific headaches were recruited for this study. Each patient did not have an allergy or reaction to aspirin, had not been diagnosed with kidney disease or liver disease, were not pregnant or planning to be pregnant within the following two months, had not been breastfeeding within the preceding two months, and had not used aspirin for the preceding 7 days.

In Period I, patients waited until they had a headache. Each patient was asked to rate his or her headache on the 10-point scale (see FIG. 3). The patient then swallowed 891 mg of aspirin (11 tablets of 81 mg each) without chewing. Each patient was asked to rate his or headache again on the 10-point scale 30-minutes later.

Period II did not start until at least two days upon completion of Period I. At Period II, patients first waited until they had a headache, each patient was asked to rate his or her headache on the 10-point scale. The patient then chewed 891 mg of aspirin (11 tablets of 81 mg each) and rubbed the aspirin in their oral mucosa, lips, gums and mouth and was asked to be careful not to swallow any of the aspirin, but to wash out their mouth afterwards with a sip of soda and spitting out such that no aspirin was swallowed. Each patient was asked to rate his or headache again on the 10-point scale 30-minutes later.

Not until at least two days later did Period III start. At Period III, patients first waited until they had a headache, and each patient was asked to rate his or her headache on the 10-point scale. The patient then chewed 891 mg of aspirin (11 tablets of 81 mg each) and rubbed the aspirin in their oral mucosa, lips, gums and mouth for about 10 seconds or until half was dissolved, then to take a sip of soda to wash the rest, but this time to swallow the soda and aspirin such that half was swallowed. Each patient was asked to rate his or headache again on the 10-point scale 30-minutes later.

As shown in FIG. 4, patients had the largest decrease during Period III had the largest decrease in headache (86%), followed by Period II (57%) and then by Period I (53%). As the total amount of aspirin was the same among Period II-IV, this example therefore demonstrates the synergistic effect between intraorally released aspirin and GI-released aspirin.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1-24. (canceled)

25. A tablet comprising:
a first portion comprising a first amount of aspirin formulated so as to, upon administration to a subject, disintegrate or dissolve intraorally; and
a second portion comprising a second amount of aspirin and a painkiller,
wherein the second portion is formulated to be substantially more difficult than the first portion to disintegrate or dissolve intraorally but is ingestible and releasable in the gastrointestinal track of the subject.

26. The tablet of claim 25, wherein the painkiller is selected from the group consisting of a non-steroidal anti-inflammatory drug (NSAID), 418 a COX-2 inhibitor, an opioid, an anxiolytic, a muscle relaxant, a methylxanthine, a salicylate, magnesium, tripatans, ergots, an anti-nausea agent, an anti-depressant, and a selective serotonin reuptake inhibitor (SSRI).

27. The tablet of claim 26, wherein the painkiller is selected from the group consisting of acetaminophen, butalbital, codeine, hydrocodone, oxycodone, pentazocine, dextropropoxyphene, propoxyphene, amitriptyline, carbamazepine, gabapentin, pregabalin and flupirtine.

28. The tablet of claim 25, further comprising caffeine.

29. A tablet comprising:
a first portion comprising a first amount of aspirin formulated so as to, upon administration to a subject, disintegrate or dissolve intraorally; and
a second portion comprising a second amount of aspirin and an agent suitable for treating a cardiovascular disease or condition,
wherein the second portion is formulated to be substantially more difficult than the first portion to disintegrate or dissolve intraorally but is ingestible and releasable in the gastrointestinal track of the subject.

30. The tablet of claim 29, wherein the agent is selected from the group consisting of a beta blocker, an ACE inhibitor, a statin, an aldosterone, a calcium channel blocker, metformin, sulfonylurea, a DPP4 inhibitor, fibrate, an anticoagulant, and eztimibe.

31. The tablet of claim 30, wherein the agent is selected from the group consisting of dipyridamole, pravastatin, metoprolol, carvedilol, captopril, zofenopril, enalapril, ramipril, quinapril, perindopril, lisinopril, benazepril, imidapril, trandolapril, fosinopril, eplerenone, warfarin, acenocoumarol, atromentin, brodifacoum, phenindone, low molecular weight heparin, fondaparinux, idraparinux, rivaroxaban, apixaban, dabigatran, hirudin, lepirudin, and bivalirudin.

32. The tablet of claim 25, wherein the first amount of aspirin constitutes at least about 20% of the sum of the first amount and second amount.

33. The tablet of claim 25, wherein the sum of the first amount and second amount is less than about 300 mg.

34. The tablet of claim 33, wherein the sum of the first amount and second amount is less than about 75 mg.

35. The tablet of claim 25, wherein the sum of the first amount and second amount is from about 300 mg to about 1000 mg.

36. The tablet of claim 35, wherein the sum is about 650 mg.

37. The tablet of claim 25, the second portion is enclosed within the first portion in the tablet.

38. The tablet of claim 25, wherein the second amount of aspirin and the agent are mixed in the second portion.

39. The tablet of claim 25, wherein the second amount of aspirin and the agent are separate in the second portion.
